(19) United States
(12) Patent Application Publication
(10) Pub. No.: US 2017/0053060 A1
(43) Pub. Date: Feb. 23, 2017

(54) METHOD FOR PREDICTING PROGNOSIS OF CANCER

(71) Applicant: INDUSTRY-ACADEMIC COOPERATION FOUNDATION, YONSEI UNIVERSITY, Seodaemun-gu, Seoul (KR)

(72) Inventors: Sang Hyun PARK, Seoul (KR); Hyun Jin KIM, Seoul (KR); Jae Gyoon AHN, Seoul (KR); Chi Hyun PARK, Seoul (KR); Young Mi YOON, Seoul (KR)

(21) Appl. No.: 15/118,817

(22) PCT Filed: Jan. 9, 2015
(86) PCT No.: PCT/KR2015/000228

§ 371 Date: Aug. 12, 2016

(30) Foreign Application Priority Data
Feb. 18, 2014 (KR) 10-2014-0018759

(51) Int. Cl. G06F 19/18; C12Q 1/68; C40B 30/02

(52) U.S. Cl. CPC G06F 19/18; C40B 30/02; C12Q 1/6886; C12Q 2600/118; C12Q 2600/158

(57) ABSTRACT

Disclosed is a method for predicting cancer prognosis, comprising: forming gene pairs by using a plurality of genes to be tested; determining clusters for the formed gene pairs through a clustering method; calculating a distribution of each gene pair based on the determined cluster; and selecting reference gene pairs for determining a class based on the calculated distribution.

### TECHNICAL FIELD

The present invention relates to a method for predicting cancer prognosis, and more particularly, to a method for predicting cancer prognosis capable of more accurately predicting the prognosis for a cancer gene by reflecting diversity of each gene through clustering in each class of the cancer.

### BACKGROUND ART

Prostate cancer is one of common cancers which occur in men as malignant tumors generated in prostate gland. In US, the prostate cancer commonly occurs in the men next to skin cancer.

In the case of most of prostate cancer, a progress speed is not fast and the cancer itself is not dangerous. Accordingly, in the case of prostate cancer patients usually over 70 years old, when examining the prognosis up to 15 years, a probability to be died is higher due to other reasons than the prostate cancer.

When the prostate cancer has not spread to other parts because the pain is severely felt or a special symptom is not shown, the patient does not easily determine whether the patient has the cancer. If a symptom of the cancer is discovered, a probability that the cancer spreads to other parts is high.

When the cancer has spread from the prostate gland to other parts, the cancer at the spread parts needs to be more worried than the prostate cancer in which the progress speed is slow. The cancer spread to other parts may have a fast progress speed, penetrate to the vital organs, and have a largely bad effect on the health of the patient.

As such, in the cancer, prognosis problems for how the current cancer will be proceeded and how much metastasis probability is present are more important than a diagnosis problem of ‘whether the cancer is or not’ according to a kind of cancer.

As the prior art related with the present invention, there is Korea Patent Application Publication No. 10-2011-0101124 (Sep. 15, 2011, published, title of invention: method for collecting data for providing information required for predicting cancer, diagnosing cancer, and verifying cancer metastasis or prognosis and kit thereof).

### SUMMARY OF THE INVENTION

In the related art, in most of methods for predicting prognosis of cancer by using gene expression levels, classification was performed based on genes in which gene expression levels are different in aggressive cancer and non-aggressive cancers.

The classifying method may be a good method for cancer diagnosis as a method used when classifying a normal sample and a tumor sample, but has a problem in that reliability is deteriorated in prognosis of determining whether the same cancer is aggressive or not.

In order to improve reliability, methods using a relationship between genes have been researched, but the methods are not correctly classified by completely reflecting heterogeneous characteristics of data.

The present invention has been made in an effort to provide a method for predicting cancer prognosis and more particularly, to a method for predicting cancer prognosis capable of more accurately predicting the prognosis for a cancer gene by reflecting diversity of each gene through clustering in each class of the cancer.

An exemplary embodiment of the present invention provides a method for predicting cancer prognosis, including: forming gene pairs by using a plurality of genes to be tested; determining clusters for the formed gene pairs through a clustering method; calculating a distribution of each gene pair based on the determined cluster; and selecting reference gene pairs for determining a class based on the calculated distribution.

According to an exemplary embodiment of the present invention, the method for predicting the cancer prognosis may further include selecting a plurality of genes to be tested in microarray data according to a predetermined reference, before forming the gene pairs.

According to an exemplary embodiment of the present invention, in the selection of the genes, the plurality of genes to be tested may be selected by using at least one of Relief-A and Symmetrical Uncertainty algorithms.

According to an exemplary embodiment of the present invention, the method for predicting the cancer prognosis may further include receiving a correct answer class for the plurality of genes to be tested, before forming the gene pairs.

According to an exemplary embodiment of the present invention, in the determining of the clusters for the formed gene pairs, the clusters may be determined by clustering for the gene pairs which belong to the same correct answer class.

According to an exemplary embodiment of the present invention, in the calculating of the distribution of each gene pair, the distribution may be calculated by a sum of Euclidean distances for average values of the determined clusters for the gene pairs.

According to an exemplary embodiment of the present invention, the method for predicting the cancer prognosis may further include; receiving expression levels for the gene pairs of the test sample, after selecting the reference gene pairs for determining the class; and predicting a class for each gene pair of the test sample by projecting the expression levels for the gene pairs of the test sample to a 2D image for the reference gene pairs.

According to an exemplary embodiment of the present invention, in the predicting of the class for each gene pair of the test sample, the class for each gene pair may be predicted based on the expression levels for the gene pairs of the test sample projected to the 2D image and Euclidean distances between the plurality of classes.

According to an exemplary embodiment of the present invention, in the predicting of the class for each gene pair of the test sample, the class for each gene pair of the test sample may be predicted as a class having a relatively smaller Euclidean distance.

According to an exemplary embodiment of the present invention, in the predicting of the class for each gene pair of the test sample, when the Euclidean distances between the gene pairs of the test sample and the plurality of classes are the same as each other, the class for each gene pair may be predicted based on a sum of the Euclidean distances between the gene pairs of the test sample and all clusters which belong to each of the plurality of classes.

According to an exemplary embodiment of the present invention, in the predicting of the class for each gene pair of the test sample, the class for each gene pair of the test sample may be predicted as a class having a relatively smaller sum of the Euclidean distances.

According to an exemplary embodiment of the present invention, the method for predicting the cancer prognosis may further include determining a final class of the test sample, after predicting the class for each gene pair of the test sample.

According to an exemplary embodiment of the present invention, in the determining of the final class of the test sample, the final class may be determined as the most predicted class among the predicted classes for each gene pair of the test sample.

According to exemplary embodiments of the present invention, it is possible to more accurately predict the prognosis of a cancer gene by reflecting diversity of each of genes through clustering in each class of the cancer.

Further, according to an exemplary embodiment of the present invention, it is possible to reflect association of a plurality of genes by determining a cluster for gene pairs.

Further, according to an exemplary embodiment of the present invention, it is possible to obtain results within a short time by selecting and testing a gene suitable for the test other than all genes of the genome.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

FIG. 1 is a functional block diagram of an apparatus for implementing a method for predicting prognosis of cancer according to an exemplary embodiment of the present invention.

FIG. 2 is a flowchart for describing a process for implementing a method for predicting prognosis of cancer according to an exemplary embodiment of the present invention.

It should be understood that the appended drawings are not necessarily to scale, presenting a somewhat simplified representation of various features illustrative of the basic principles of the invention. The specific design features of the present invention as disclosed herein, including, for example, specific dimensions, orientations, locations, and shapes will be determined in part by the particular intended application and use environment.

In the figures, reference numbers refer to the same or equivalent parts of the present invention throughout the several figures of the drawing.

### DETAILED DESCRIPTION

Hereinafter, a method for predicting prognosis of cancer according to an exemplary embodiment of the present invention will be described in detail with reference to the accompanying drawings. Thicknesses of lines, sizes of constitute elements, and the like illustrated in the drawings in this process can be exaggerated for clarity and convenience of the description. In addition, terms to be described below may vary according to user's and operator's intentions, the convention, or the like as terms defined by considering functions of the present invention. Therefore, the definition should be made according to the contents throughout this specification.

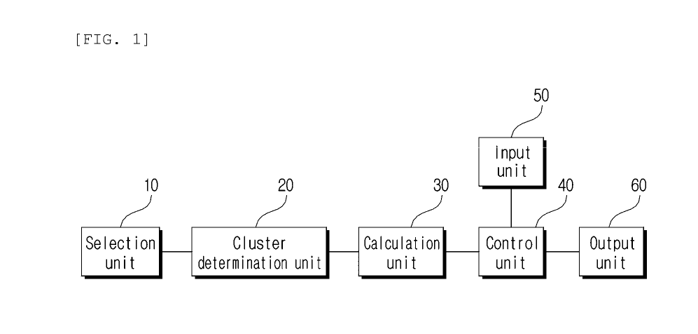

FIG. 1 is a functional block diagram of an apparatus for implementing a method for predicting prognosis of cancer according to an exemplary embodiment of the present invention.

Referring to FIG. 1, the apparatus for implementing the method for predicting the prognosis of cancer includes a selection unit 10, a cluster determination unit 20, a calculation unit 30, a control unit 40, an input unit 50, and an output unit 60.

The selection unit 10 selects a plurality of genes to be tested for predicting the cancer prognosis from microarray data according to a predetermined reference.

The microarray data mean data in an array form which represent respective expression levels for the plurality of genes of the genome.

The microarray data include thousands to tens of thousands pieces of data, and when the number of data for each gene is not reduced, it takes a too long execution time to perform the following process for predicting the cancer prognosis, and thus there is a problem in that time complexity is large.

Accordingly, in the exemplary embodiment, the selection unit 10 selects the plurality of genes to be tested according to the predetermined reference so as to use only the data for a predetermined number of genes in the entire data.

In detail, the selection unit 10 selects the plurality of genes to be tested by using at least one of Relief-A or Symmetrical Uncertainty algorithms.

The Relief-A is an algorithm of selecting a characteristic on the assumption that as any characteristic has a similar value between the plurality of genes which belong to the same class and has different values between the plurality of genes which belong to different classes, the corresponding characteristic is a good characteristic.

Further, the Symmetrical Uncertainty is an algorithm of selecting a characteristic on the assumption that as dependence on any characteristic and the class is increased, the corresponding characteristic is a good characteristic.

Since the Relief-A and Symmetrical Uncertainty algorithms are techniques which are previously known, the detailed description of the implementation process will be not described.

As such, in the exemplary embodiment, only genes which are predicted to have a meaning are selected from many genes by using at least one of the aforementioned Relief-A and Symmetrical Uncertainty to be tested.

Accordingly, in the exemplary embodiment, since the predetermined number of genes is selected by the selection unit 10, time complexity of the test may be reduced. In addition, since worthless genes for the classification may be excluded, classification accuracy may be improved.

The cluster determination unit 20 determines a cluster for the plurality of genes through a clustering method.

The clustering method is an analysis method of grouping any object or targets into several clusters so that objects having similarity or similar characteristics by a distance are grouped together.

That is, in the exemplary embodiment, respective clusters are divided by clustering the plurality of genes to be tested.

Particularly, in the exemplary embodiment, the cluster determination unit 20 performs 2-dimensional clustering of forming gene pairs by using the plurality of genes to be tested and determining the clusters for the formed gene pairs.

As such, in the exemplary embodiment, the cluster determination unit 20 may reflect association of the plurality of genes by determining the clusters for the gene pairs, not determining the clusters for the plurality of genes.

Further, in the exemplary embodiment, the cluster determination unit 20 determines a cluster for the gene pair through clustering in a class which is clustering for the gene pair belonging to the same class, not clustering between classes.

When performing a general clustering, the clustering is performed on the assumption that genes in different classes have different clusters, and thus heterogeneity in one class is ignored and a false positive or false negative result may be shown.

Accordingly, in the exemplary embodiment, a cluster for the gene pair is more accurately determined through clustering in the class assuming that the clusters may be different even in the genes in the same class.

In addition, to this end, the cluster determination unit 20 receives a correct answer class for the plurality of genes and performs the clustering for the gene pair which belongs to the same assumed class.

In this case, in the exemplary embodiment, the correct answer class for the plurality of genes may be distinguished and input by a class distinguishing a normal class and a cancer patient class, a class distinguishing a high-aggressive cancer patient class and a low-aggressive cancer patient class, or the like.

That is, in the exemplary embodiment, the cluster determination unit 20 receives correct answer classes classified according to an existing technique or doctor's determination and determines a more specific and accurate cluster through the clustering in the corresponding class.

In addition, as described above, in the case where the correct answer class distinguishing the normal class and the cancer patient class is input, the cluster determination unit 20 determines a cluster through clustering in a 2D class for the gene pair formed by using the plurality of genes to distinguish the genes belonging to the cancer patient class into a cluster which belongs to a dangerous cancer having high aggression and a less dangerous cancer having low aggression.

In this case, when n genes are selected by the selection unit 10, gene pairs which may be formed by the n genes are n(n−1)/2 and the clustering is also performed n(n−1)/2 times with respect to each gene pair.

In addition, as a clustering method according to the exemplary embodiment, a K-means algorithm may be used. The K-means algorithm is a clustering algorithm based on a distance of decomposing n objects into K clusters to ensure some reasonable execution time even in the case where the number of genes is large due to a fast execution time.

However, in the exemplary embodiment, the cluster determination unit 20 needs not to perform the clustering by using only the K-means algorithm, but may perform clustering for gene pairs by using various clustering methods which are not described.

The calculation unit 30 calculates a distribution of gene pairs based on the cluster determined by the cluster determination unit 20.

According to the exemplary embodiment, in order to predict a class and a cluster of a sample patient, values of all gene pairs of the patient are projected to a 2D image to be classified to a class of the closest cluster.

In this case, in order to predict the class of the sample patient, when n genes are selected, as described above, since each class is predicted with respect to a total of n(n−1)/2 gene pairs, the predicting classes also become n(n−1)/2.

When using all predicted classes for many genes, a long execution time is taken and a clustering result for gene pairs which are not suitable for classification may be included.

Accordingly, in the exemplary embodiment, the calculation unit 30 calculates a distribution of each gene pair based on a cluster for a gene pair determined by the cluster determination unit 20 in order to select the gene pair suitable for the class classification.

In detail, as each cluster is independently present without overlapping, the genes of the sample patient may be accurately distinguished and thus, in the exemplary embodiment, the gene pair that is the reference of the class classification is selected based on the distribution of each gene pair.

Particularly, the calculation unit 30 calculates a distribution of each gene pair by a sum of Euclidean distances for clusters determined for each gene pair.

Particularly, when K clusters are present every class, a 2D image coordinate of an average value of an a-th cluster in a first class is (x1a, y1a), and a 2D image coordinate of an average value of a b-th cluster in a second class is (x2a, y2a), a distribution d may be calculated through the following Equation.

$$d = {\sum }_{a = 1}^{K} {\sum }_{b = 1}^{K} [ {( {x}_{1 a} - {x}_{2 b} )}^{2} + {( {y}_{1 a} - {y}_{2 b} )}^{2} ]$$

The control unit 40 selects reference gene pairs for determining a class based on the distribution of each gene pair calculated by the calculation unit 30. In this case, the number of reference gene pairs for determining the class may vary according to the user's selection.

Through the aforementioned process, the control unit 40 may learn a reference value for determining the class which belongs to a specific genome by using the microarray data.

In addition, in the following process, the control unit 40 may accurately determine which class a test sample belongs to through a comparison with the aforementioned reference gene pair when a specific test sample is input.

To this end, the control unit 40 receives gene pairs of the test sample from the input unit 50.

In addition, the control unit 40 may predict a class for each gene pair of the test sample by projecting values of the gene pairs of the test sample to the 2D image for the reference gene pair.

To this end, the control unit 40 primarily predicts a class for each gene pair based on the Euclidean distances between each gene pair of the test sample projected to the 2D image and the plurality of classes.

Particularly, the control unit 40 predicts a class PC(S) for each gene pair through the following Equation.

$$PC ( S ) = \{ \begin{matrix}Class 1 , & if {ud}_{min} ( C 1 ) < {ud}_{min} ( C 2 ) \\ Class 2 , & if {ud}_{min} ( C 1 ) > {ud}_{min} ( C 2 )\end{matrix}$$

(In this case, udₘᵢₙ(Ci) means the smallest Euclidean distance between the test sample and a class Ci.)

That is, the class of the gene pair of the test sample is predicted as a class of which the Euclidean distance between the gene pair and the class of the test sample is relatively smaller.

However, in this case, among the gene pairs, with respect to a cluster in a different class, like udₘᵢₙ(C1)=udₘᵢₙ(C2), gene pairs having the same smallest distance may be present.

In this case, the control unit 40 secondarily predicts a class for each gene pair based on a sum of Euclidean distances between the gene pairs of the test sample and all clusters which belong to the plurality of classes.

Particularly, the control unit 40 predicts a class for each gene pair through the following Equation.

$$PC ( S ) = \{ \begin{matrix}Class 1 , & if ud ( C 1 ) < ud ( C 2 ) \\ Class 2 , & if ud ( C 1 ) > ud ( C 2 )\end{matrix}$$

(In this case, ud(Ci) means a sum of Euclidean distances between the test sample and all clusters of a specific class Ci.)

In this case, the class of the gene pair of the test sample is predicted, as a class of which a sum of Euclidean distances between the gene pairs of the test sample and all clusters which belong to the plurality of classes is relatively smaller.

If the control unit 40 selects m reference gene pairs for determining the class, m class prediction results for the gene pairs of the test sample are present.

The control unit 40 determines the final class of the test sample by using the m class prediction results. Particularly, the final class is determined as the most predicted class among predicted classes for the gene pairs of the test sample.

The output unit 60 outputs the final class determined by the control unit 40 in a form which may be verified by the user.

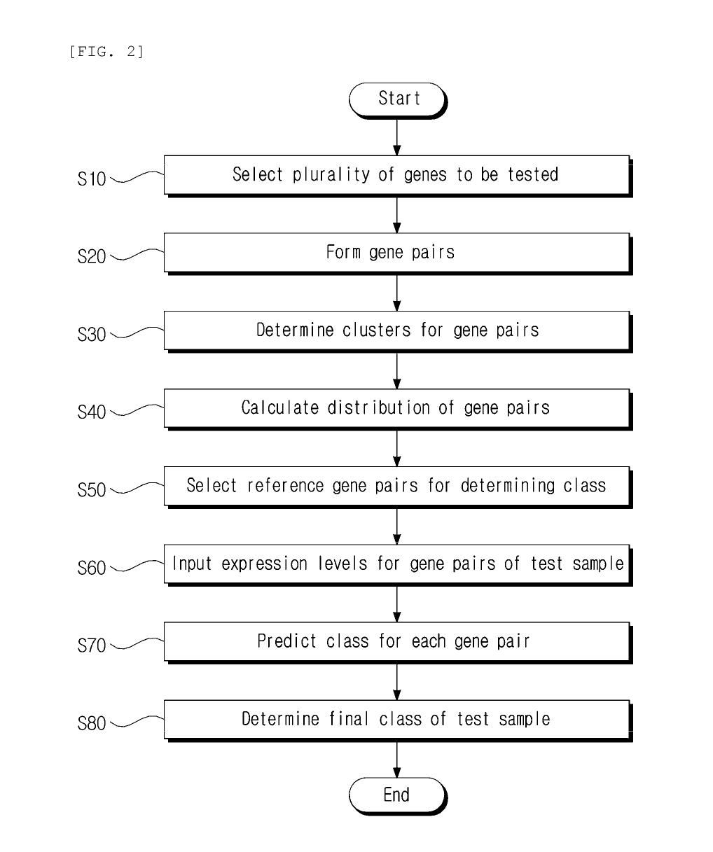

FIG. 2 is a flowchart for describing a process for implementing a method for predicting prognosis of cancer according to an exemplary embodiment of the present invention.

Referring to FIG. 2, when describing a process for implementing the method for predicting the prognosis of cancer according to the exemplary embodiment of the present invention, first, the selection unit 10 selects a plurality of genes to be tested from the microarray data according to a predetermined reference (S10).

The microarray data include thousands to tens of thousands pieces of data, and when the number of data for each gene is not reduced, it takes a too long execution time to perform the following process for predicting the cancer prognosis, and thus there is a problem in that time complexity is large.

Accordingly, in the exemplary embodiment, the selection unit 10 selects the plurality of genes to be tested according to the predetermined reference so as to use only the data for a predetermined number of genes in the entire data.

In detail, the selection unit 10 selects the plurality of genes to be tested by using at least one of Relief-A or Symmetrical Uncertainty algorithms. The Relief-A and Symmetrical Uncertainty algorithms are previous known algorithms and thus the detailed description will be not described.

As such, in the exemplary embodiment, since the predetermined number of genes is selected by the selection unit 10, time complexity of the test may be reduced. In addition, since worthless genes for the classification may be excluded, classification accuracy may be improved.

In addition, the cluster determination unit 20 forms gene pairs by using the plurality of genes to be tested (S20), which is selected by the selection unit 10 in the aforementioned step (S10) and determines clusters for the formed gene pairs through the clustering method (S30).

As such, in the exemplary embodiment, the cluster determination unit 20 may reflect association of the plurality of genes by determining the clusters for the gene pairs, not determining the clusters for the plurality of genes.

Further, in the exemplary embodiment, the cluster determination unit 20 determines a cluster for the gene pair through clustering in a class which is clustering for the gene pair belonging to the same class, not clustering between classes.

When performing a general clustering, the clustering is performed on the assumption that genes in different classes have different clusters, and thus heterogeneity in one class is ignored and a false positive or false negative result may be shown.

Accordingly, in the exemplary embodiment, a cluster for the gene pair is more accurately determined through clustering in the class assuming that the clusters may be different even in the genes in the same class.

In addition, to this end, the cluster determination unit 20 receives a correct answer class for the plurality of genes and performs the clustering for the gene pair which belongs to the same correct answer class.

Subsequently, the calculation unit 30 calculates the distribution of each gene pair based on the cluster determined by the aforementioned step (S30) (S40) and the control unit 40 selects reference gene pairs for determining the class based on the calculated distribution (S50).

According to the exemplary embodiment, in order to predict a class and a cluster of a sample patient, values of all gene pairs of the patient are projected to a 2D image to be classified to a class of the closest cluster.

In this case, in order to predict the class of the sample patient, when n genes are selected, as described above, since each class is predicted with respect to a total of n(n−1)/2 gene pairs, the predicting classes also become n(n−1)/2.

When using all predicted classes for many genes, a long execution time is taken and a clustering result for gene pairs which are not suitable for classification may be included.

Accordingly, in the exemplary embodiment, in order to select the gene pair suitable for the class classification, the calculation unit 30 calculates a distribution of each gene pair based on the cluster for the gene pair determined in the aforementioned step (S30).

In detail, as each cluster is independently present without overlapping, the genes of the sample patient may be accurately distinguished and thus, in the exemplary embodiment, the gene pair that is the reference of the class classification is selected based on the distribution of each gene pair.

As an example, the distribution of each gene pair may be calculated by a sum of Euclidean distances for average values determined for each gene pair, but is not limited thereto, and the distribution of each gene pair may be calculated through various methods.

Next, when the gene pair of the test sample for determining the class is input by the input unit 50 (S60), the control unit 40 predicts the class for each gene pair (S70).

Particularly, the control unit 40 may predict a class for each gene pair of the test sample by projecting values of the gene pairs of the test sample to the 2D image for the reference gene pair.

To this end, the control unit 40 primarily predicts a class for each gene pair based on the Euclidean distance between each gene pair of the test sample projected to the 2D image and the plurality of classes.

Particularly, the control unit 40 predicts a class PC(S) for each gene pair through the following Equation.

$$PC ( S ) = \{ \begin{matrix}Class 1 , & if {ud}_{min} ( C 1 ) < {ud}_{min} ( C 2 ) \\ Class 2 , & if {ud}_{min} ( C 1 ) > {ud}_{min} ( C 2 )\end{matrix}$$

(In this case, udₘᵢₙ(Ci) means the smallest Euclidean distance between the test sample and a class Ci.)

That is, the class of the gene pair of the test sample is predicted as a class of which the Euclidean distance between the gene pair and the class of the test sample is relatively smaller.

However, in this case, among the gene pairs, with respect to a cluster in a different class, like udₘᵢₙ(C1)=udₘᵢₙ(C2), gene pairs having the same smallest distance may be present.

In this case, the control unit 40 secondarily predicts a class for each gene pair based on a sum of Euclidean distances between the gene pairs of the test sample and all clusters which belong to the plurality of classes.

Particularly, the control unit 40 predicts a class for each gene pair through the following Equation.

$$PC ( S ) = \{ \begin{matrix}Class 1 , & if ud ( C 1 ) < ud ( C 2 ) \\ Class 2 , & if ud ( C 1 ) > ud ( C 2 )\end{matrix}$$

(In this case, ud(Ci) means a sum of Euclidean distances between the test sample and all clusters of a specific class Ci.)

That is, the class of the gene pair of the test sample is predicted, as a class of which a sum of Euclidean distances between the gene pairs of the test sample and all clusters which belong to the plurality of classes is relatively smaller.

In addition, the control unit 40 determines a final class of the test sample by using the class for each gene pair of the test sample predicted in the aforementioned step (S70) (S80).

Particularly, the final class is determined as the most predicted class among predicted classes for the gene pairs of the test sample.

According to the exemplary embodiments of the present invention, it is possible to more accurately predict the prognosis of a cancer gene by reflecting diversity of each gene through clustering in each class of the cancer.

Further, according to the exemplary embodiments of the present invention, it is possible to reflect association of a plurality of genes by determining a cluster for gene pairs.

Further, according to the exemplary embodiments of the present invention, it is possible to obtain results within a short time by selecting and testing a gene suitable for the test other than all genes of the genome.

As described above, the exemplary embodiments have been described and illustrated in the drawings and the specification. The exemplary embodiments were chosen and described in order to explain certain principles of the invention and their practical application, to thereby enable others skilled in the art to make and utilize various exemplary embodiments of the present invention, as well as various alternatives and modifications thereof. As is evident from the foregoing description, certain aspects of the present invention are not limited by the particular details of the examples illustrated herein, and it is therefore contemplated that other modifications and applications, or equivalents thereof, will occur to those skilled in the art. Many changes, modifications, variations and other uses and applications of the present construction will, however, become apparent to those skilled in the art after considering the specification and the accompanying drawings. All such changes, modifications, variations and other uses and applications which do not depart from the spirit and scope of the invention are deemed to be covered by the invention which is limited only by the claims which follow.

What is claimed is:

1. A method for predicting cancer prognosis, the method comprising:
forming gene pairs by using a plurality of genes to be tested;
determining clusters for the formed gene pairs through a clustering method;
calculating a distribution of each gene pair based on the determined cluster; and
selecting reference gene pairs for determining a class based on the calculated distribution.

2. The method for predicting the cancer prognosis of claim 1, the method further comprising:
selecting a plurality of genes to be tested in microarray data according to a predetermined reference, before forming the gene pairs.

3. The method for predicting the cancer prognosis of claim 2, wherein in the selection of the genes, the plurality of genes to be tested is selected by using at least one of Relief-A and Symmetrical Uncertainty algorithms.

4. The method for predicting the cancer prognosis of claim 1, the method further comprising:
receiving a correct answer class for the plurality of genes to be tested, before forming the gene pairs.

5. The method for predicting the cancer prognosis of claim 4, wherein in the determining of the clusters for the formed gene pairs, the clusters are determined by clustering for the gene pairs which belong to the same correct answer class.

6. The method for predicting the cancer prognosis of claim 1, wherein in the calculating of the distribution of each gene pair, the distribution is calculated by a sum of Euclidean distances for average values of the determined clusters for the gene pairs.

7. The method for predicting the cancer prognosis of claim 1, the method further comprising:
receiving expression levels for the gene pairs of the test sample, after selecting the reference gene pairs for determining the class; and
predicting a class for each gene pair of the test sample by projecting the expression levels for the gene pairs of the test sample to a 2D image for the reference gene pairs.

8. The method for predicting the cancer prognosis of claim 7, wherein in the predicting of the class for each gene pair of the test sample, the class for each gene pair is predicted based on the expression levels for the gene pairs of the test sample projected to the 2D image and Euclidean distances between the plurality of classes.

9. The method for predicting the cancer prognosis of claim 8, wherein in the predicting of the class for each gene pair of the test sample, the class for each gene pair of the test sample is predicted as a class having a relatively smaller Euclidean distance.

10. The method for predicting the cancer prognosis of claim 8, wherein in the predicting of the class for each gene pair of the test sample, when the Euclidean distances between the gene pairs of the test sample and the plurality of classes are the same as each other, the class for each gene pair is predicted based on a sum of the Euclidean distances between the gene pairs of the test sample and all clusters which belong to each of the plurality of classes.

11. The method for predicting the cancer prognosis of claim 10, wherein in the predicting of the class for each gene pair of the test sample, the class for each gene pair of the test sample is predicted as a class having a relatively smaller sum of the Euclidean distances.

12. The method for predicting the cancer prognosis of claim 7, the method further comprising:
determining a final class of the test sample, after predicting the class for each gene pair of the test sample.

13. The method for predicting the cancer prognosis of claim 12, wherein in the determining of the final class of the test sample, the final class is determined as the most predicted class among the predicted classes for each gene pair of the test sample.
